(19) United States
(12) Patent Application Publication
(10) Pub. No.: US 2005/0211908 A1
(43) Pub. Date: Sep. 29, 2005

(54) Bluetooth wireless dental X-ray device and system

(75) Inventors: Francis Dieras, Bordeaux (FR); Alain Mazuir, Saint Maximin la Sainte Baume (FR)

Correspondence Address: MILLER & BARNS, PLLC, 230 EAST BROADWAY, SUITE 250, GRANVILLE, OH, 43023, US

(73) Assignee: SOPRO, La Ciotat Cedex (FR)

(21) Appl. No.: 11/136,743

(22) Filed: May 25, 2005

(30) Foreign Application Priority Data
Apr. 12, 2005 (FR) 0503632
Apr. 12, 2005 (FR) 0503633

(51) Int. Cl. G21K 1/12; H05G 1/60; A61B 6/00; G01N 23/00; G01J 1/00; G01T 1/24; H01L 25/00

(52) U.S. Cl. 250/370.09

(57) ABSTRACT

The dental X-ray image acquisition device comprises an in-mouth sensor and a control unit suitable for controlling the in-mouth sensor and for triggering the acquisition of an image, for storing it, and for transferring said image to at least one computer via digital wireless communications means in compliance with the Bluetooth (registered trademark) standard.

### BACKGROUND OF THE INVENTION

The present invention relates to the general field of dental X-ray devices and systems.

More particularly, the invention relates to an X-ray device including digital wireless communications means for transferring X-ray images or “radiographs” to a computer, for displaying and/or storage purposes.

Dental X-ray systems are known that make use of digital radiocommunications technology to transfer radiographs to a computer, e.g. for display purposes.

However presently known systems suffer from the drawback of consuming a great deal of energy and of being highly sensitive to interference.

Unfortunately, in a dentist's office, there are numerous items of equipment that are liable to produce interference, making wireless communication difficult.

### OBJECT AND SUMMARY OF THE INVENTION

A main object of the present invention is thus to mitigate those drawbacks by proposing a dental X-ray image acquisition device comprising:
an in-mouth sensor; and
a control unit suitable for controlling the in-mouth sensor to trigger image acquisition, to store the image, and to transfer the image to at least one computer via digital wireless communications means complying with the Bluetooth (registered trademark) standard as defined by the IEEE 802.15 version 2 and subsequent standards.

The device of the invention solves the above-mentioned problems, since Bluetooth (registered trademark) technology consumes very little energy and is particularly robust against interference. The acquired images can thus be displayed on various types of terminal (portable telephone, personal digital assistant (PDA), . . . ).

Traditionally, the Bluetooth standard has not been used for transferring digital images, given its data rate that is limited compared with that of wired installations, in particular.

Thus, in a preferred embodiment, the device of the invention includes a pre-display mode in which a low resolution image of the radiograph is transferred, where such transfer can take place in a length of time that is acceptable to the practitioner, with the missing information for reconstituting a high-resolution image being transferred subsequently in time that is not critical from the medical examination point of view.

In the context of dental X-rays, a particular problem arises due to the fact that the practitioner taking the X-ray cannot view the image on the computer while the radiograph is being taken.

This means that images are often lost in practice, either because of the above-mentioned interference, or because the computer that was to receive them was busy with some other task, and that can mean that the radiographs must be taken again, with the drawback of subjecting the patient to additional radiation.

In order to solve this particular problem, a preferred embodiment of the device of the invention includes monitoring means suitable:
for conserving the image in a memory if no wireless communication is properly set up with at least one computer; and
for transferring said image to at least one computer as soon as such communication is properly set up. By means of this characteristic, there is no longer any need to take additional radiographs just because a radiograph has been poorly transmitted. This characteristic also avoids a significant loss of time involved with taking pointless radiographs.

In a preferred embodiment, the monitoring means are adapted to receive a command coming from a computer via the wireless communications means, and to set parameters in the control unit on the basis of information extracted from the command.

The invention thus makes it possible, in particular, for a practitioner to personalize radiographs as a function of parameters that are specific to each patient and/or to the anatomical zone under observation.

In a preferred variant embodiment, the device of the invention includes a register for storing at least one configuration value authorizing the device to communicate with at least one computer.

This characteristic advantageously makes it possible to set up a genuine Bluetooth (registered trademark) network within a service or office that has a plurality of dentists' chairs, thereby limiting the volume occupied by equipment in a care center, and reducing the cost thereof.

In this context, it is even more important to be able to conserve a digital image in memory, in particular in network configurations where a plurality of control units are transferring their X-ray images to the same computer.

Thus, in a second aspect, the invention also provides a dental X-ray system comprising at least one dental X-ray image acquisition device as described briefly above and at least one computer having wireless communications means complying with the Bluetooth (registered trademark) standard for communicating with said device.

### BRIEF DESCRIPTION OF THE DRAWINGS

Other characteristics and advantages of the present invention appear from the following description given with reference to the accompanying drawings which show an embodiment of the invention that has no limiting character, and in which:

FIG. 1 shows a dental X-ray system in accordance with the invention in a preferred embodiment; and

FIG. 2 shows a method implemented by a dental X-ray image acquisition device in accordance with the invention.

### DETAILED DESCRIPTION OF AN EMBODIMENT

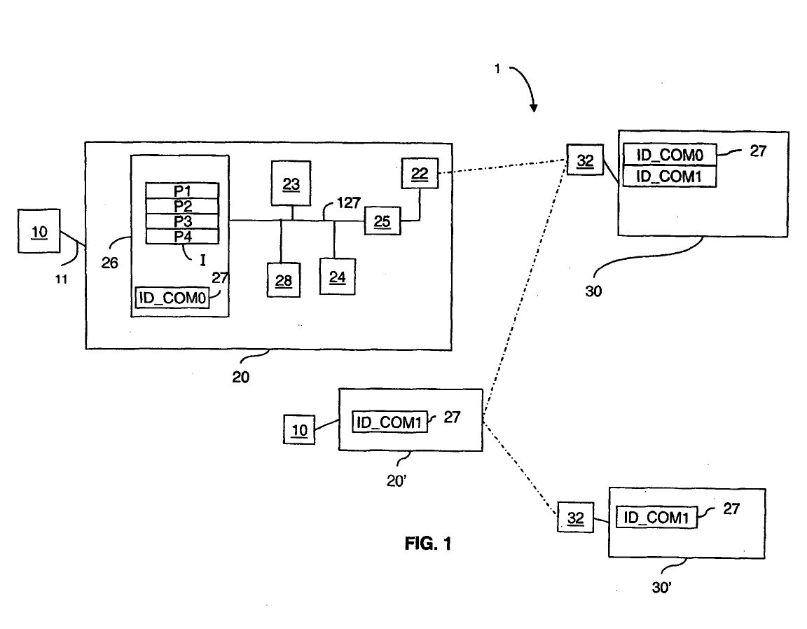

FIG. 1 shows a wireless dental X-ray system 1 in accordance with the invention in a preferred embodiment thereof.

The system comprises at least one computer 30 having digital wireless telecommunications means in compliance with the Bluetooth (registered trademark) standard, as defined by the IEEE 802.15 version 2 and subsequent standards.

In a preferred embodiment, the wireless communications means comply with the enhanced data rate (EDR) Bluetooth (registered trademark) 2.0 standard.

In the present document, the term “computer” is used to designate any digital data processor system, in particular for the purposes of displaying and/or storing dental X-ray images.

The computer 30 is suitable for using the Bluetooth (registered trademark) telecommunications means 32 to communicate with a control unit 20 connected by a cable 11 to an in-mouth sensor 10 having a charge-coupled device (CCD) sensor.

In particular, from the computer 30, the practitioner can send a command over the wireless telecommunications means 22 for configuring the control unit 20 with parameters that are specific to the patient.

The control unit 20 has means 24 adapted to generate control and monitoring signals for the CCD sensor, these signals being conveyed over the cable 11.

In a variant, the CCD sensor may be replaced by a complementary metal oxide semiconductor (CMOS) sensor.

To communicate with the computer 30, the control unit 20 includes wireless telecommunications means 22 in compliance with the Bluetooth (registered trademark) standard and compatible with the wireless telecommunications means 32 of the computer 30.

In the embodiment described herein, when the control unit 20 is switched on, a computer 30 and the control unit 20 verify in registers 27 whether they are configured with an ID_COM0 value that authorizes Bluetooth (registered trademark) communication between those elements for transferring a dental image I and for enabling it to be picked up by the computer 30.

In the example of FIG. 1:
the computer 30 is configured to communicate with control units 20 and 20′; and
the computer 30′ is configured to communicate solely with control unit 20′.

These computers 30 and 30′ may have different functions. For example, the computer 30′ may serve as a database for archiving dental X-rays for medical and legal purposes.

In known manner, the control unit 20 includes a memory 26 for storing a dental image I acquired by the sensor 10. This image may be corrected by image processor means 23 in the unit 20, for example in the event of a defective pixel.

All of the elements of the unit 20 are connected to a common bus 127 and controlled by a processor or a digital signal processor (DSP) 25.

In particular, the processor 25 is suitable for responding to a command from the computer 30 as received over the wireless means 22, by sending any portion P3 of the image I, in particular in the event of reception being poor.

The processor 25 includes means for determining whether communication has been properly set up with at least one computer 30. In practice, the processor 25 verifies that the information transmitted towards the computers 30 and 30′ with which it is in communication has been acknowledged.

The processor 25 is also suitable for switching on an indicator lamp 28, e.g. constituted by a light-emitting diode (LED) whenever communication is set up and maintained with at least one computer 30, 30′.

In accordance with the invention, the control unit 20 is suitable for conserving the dental image I in the memory 26 whenever Bluetooth (registered trademark) communication is not properly established with a computer 30, 30′ of the system 1. The system is thus “self-contained”.

This characteristic is very useful in particular if the computer 30 is switched off, inoperative, or wrongly configured.

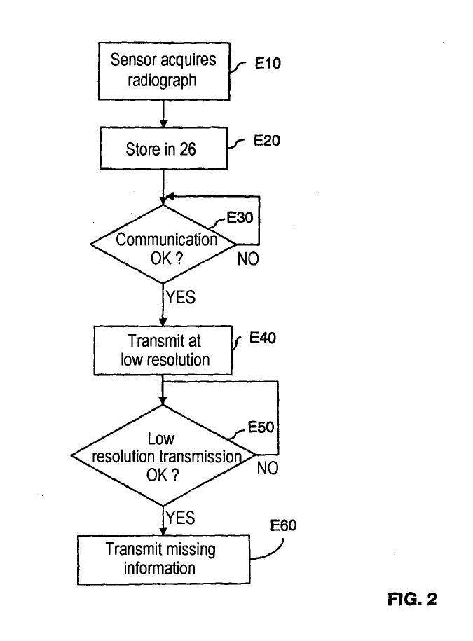

With reference to FIG. 2, the main steps of a method implemented by the image acquisition device described above with reference to FIG. 1 are described below.

When the practitioner takes a radiograph (step E10), the dental X-ray image I is stored (step E20) in the memory 26.

This storage step E20 is followed by a test E30 during which the processor 25 verifies whether Bluetooth (registered trademark) communication is properly set up with at least one computer 30, 30′ of the system 1.

Until this is the case, the result of test E30 is negative, and the processor 25 continues to look for a computer 30, 30′ on the network with which the control unit 20 can set up correctly authorized Bluetooth (registered trademark) communication.

Once communication has been set up with a computer 30, the result of test E30 becomes positive. This test is then followed by a step E40 during which a low resolution image of the radiograph is transferred over the Bluetooth (registered trademark) link.

In practice, this low resolution image can be obtained by selecting one pixel in four from the representation in memory 26 of the radiograph I. This can be referred to as “pre-display” mode.

Step E40 of transmitting the low resolution image is followed by a step E50 during which the processor 25 verifies that the low resolution image has been received in full by the computer 30.

In practice, the processor 25 verifies, for example, that all of the portions of the low resolution image as transmitted in succession to the computer 30 have been acknowledged. If not, the missing portions of the low resolution image are repeated.

Once the low resolution image has been transmitted in full, step E50 is followed by a step E60 during which the information that is missing for reconstituting the dental image I in full is read from the memory 26 and transferred to the computer 30.

1. A device for acquiring a dental x-ray image, the device comprising:
an in-mouth sensor; and
a control unit suitable for controlling the in-mouth sensor to trigger acquisition of said image, to store it, and to transfer said image to at least one computer via digital wireless communications means, wherein said wireless communications means comply with the BLUETOOTH standard.

2. A device according to claim 1, including a pre-display mode during which a low resolution image of said image is transferred initially, said initial transfer requiring a length of time that is acceptable for a practitioner, with the information that is missing for reconstituting said image in full being transferred subsequently in time that is not critical from the medical examination point of view.

3. A device according to claim 1, including control means suitable:
for conserving said image in a memory so long as wireless communication is not properly established with said at least one computer; and
for transferring said image to said at least one computer as soon as said communication is properly established.

4. A device according to claim 3, wherein said control means are suitable for receiving a command coming from said computer via said wireless communications means, and for setting parameters of said control unit on the basis of information extracted from said command.

5. A device according to claim 1, including a register for storing at least one configuration value authorizing said device to communicate with said at least one computer.

6. (canceled)

7. A device according to claim 1, further comprising:
at least one computer having wireless communications means in compliance with the BLUETOOTH standard for communicating with said device.

8. A device according to claim 2, further comprising:
at least one computer having wireless communications means in compliance with the BLUETOOTH standard for communicating with said device.

9. A device according to claim 3, further comprising:
at least one computer having wireless communications means in compliance with the BLUETOOTH standard for communicating with said device.

10. A device according to claim 4, further comprising:
at least one computer having wireless communications means in compliance with the BLUETOOTH standard for communicating with said device.

11. A device according to claim 5, further comprising:
at least one computer having wireless communications means in compliance with the BLUETOOTH standard for communicating with said device.

12. A device for acquiring a dental X-ray image, the device comprising:
an in-mouth sensor; and
a control unit to trigger acquisition of an image by the in-mouth sensor, to store said image, and to transfer said image to at least one computer, wherein said transfer is a digital wireless communication in compliance with the BLUETOOTH standard.

13. A device according to claim 12, including a pre-display mode during which an initial transfer of a low resolution image of said image, said initial transfer requiring a first length of time, and a subsequent transfer of a high resolution image in a second length of time, said second length of time being longer than said first length of time.

14. A device according to claim 12, including memory conserving said image so long as wireless communication is not properly established with said at least one computer; and
transferring said image to said at least one computer as soon as said communication is properly established.

15. A device according to claim 14, wherein said control unit receives a wireless transmission of a command from said computer, and for setting parameters of said control unit on the basis of information extracted from said command.

16. A device according to claim 12, including a register storing at least one configuration value authorizing said device to communicate with said at least one computer.

17. A system for acquiring a dental X-ray image, comprising:
an in-mouth sensor;
at least one computer for receiving an image; and
a control unit to trigger acquisition of an image by the in-mouth sensor, to store said image, and to transfer said image to said at least one computer, wherein said transfer is a digital wireless communication in compliance with the IEEE 802.15 standard.

18. The system according to claim 17, wherein said control unit further comprises:
a memory to store said image so long as wireless communication is not properly established with said at least one computer; and to transfer said image to said at least one computer as soon as said communication is properly established.

19. The system according to claim 18, wherein said control unit receives a wireless command from said computer setting parameters of said control unit on the basis of information extracted from said command.

20. The system according to claim 17, further comprising:
a register for storing at least one configuration value authorizing said device to communicate with said at least one computer.

21. The system according to claim 17, further comprising:
a display.
